(19) United States
(12) Patent Application Publication
(10) Pub. No.: US 2012/0243667 A1
(43) Pub. Date: Sep. 27, 2012

(54) LIGHT FOR X-RAY IMAGING SYSTEM

(76) Inventor: Daniel A. Walters, Salt Lake City, UT (US)

(21) Appl. No.: 13/071,743

(22) Filed: Mar. 25, 2011

(51) Int. Cl. A61B 6/08

(52) U.S. Cl. 378/206

(57) ABSTRACT

Systems and methods for making and using a light with an X-ray imaging arm are described. The light can be configured to have a light source that is disposed around at least a portion of a perimeter of an X-ray imaging arm's X-ray source and/or X-ray detector. The light source can be positioned to direct its light to a field of view that is located between the X-ray source and the X-ray detector. The light can comprise a light source, a switching mechanism, and wiring to connect the light source and switching mechanism to a power source, such as a battery. The light can be electrically connected into the imaging arm or can be part of an accessory that can be retrofitted to the imaging arm. Other embodiments are described.

### FIELD

This application relates generally to X-ray equipment. More specifically, this application relates to systems and methods for providing a lighting system that illuminates a field of view between an X-ray source and an X-ray detector on an X-ray imaging system.

### BACKGROUND

A typical X-ray imaging system comprises an X-ray source and an X-ray detector. X-rays emitted from the X-ray source can impinge on the X-ray detector and provide an X-ray image of the object or objects that are placed between the X-ray source and the detector. In one type of X-ray imaging system, a fluoroscopic imaging system, the X-ray detector is often an image intensifier or more recently a flat panel digital detector.

Fluoroscopic imaging systems can be either mobile or fixed. Mobile fluoroscopic imaging systems are used in a variety of clinical environments, such as radiology and surgery departments of a medical facility. Fixed fluoroscopic imaging systems contains a gantry that is secured to a floor, wall or ceiling. In an attempt to improve mobile 3D image quality, a mobile system has been introduced that fully encloses the rotational gantry into an “0” shape. Mobile fluoroscopic imaging systems may include C-arms, O-arms, L-arms or other imaging assembly.

In some configurations, the C-arm assembly remains stationary relative to a subject for single angle imaging. In other configurations, the C-arm assembly moves relative to the subject in order to acquire images from multiple angles. In some arrangements, the C-arm assembly is manually repositioned to generate images from different angles. In other arrangements, the C-arm assembly is moved along a predetermined path by operation of a motorized drive mechanism in order to generate images from multiple angles.

In addition to the X-ray source and the X-ray detector, the typical fluoroscopic imaging system comprises a main assembly, a support assembly, and a gantry or arm assembly. The main assembly is coupled to the movable support assembly and the support assembly supports the movable gantry or arm assembly. The X-ray source and the X-ray detector are positioned opposite each other on the gantry or positioned at opposite ends of the arm assembly. For mobile imaging systems, the main assembly typically includes wheels for moving and/or positioning the imaging system.

When a practitioner takes X-rays of a patient, it is desirable to take several X-rays of one or more portions of the patient's body from a number of different positions and angles, preferably without needing to frequently reposition the patient.

### SUMMARY

This application relates to X-ray equipment. In particular, this application discusses systems and methods for making and using a light for the field of view in an X-ray imaging arm. In such systems and methods, the light can be configured to have a light source that is disposed around at least a portion of a perimeter of an X-ray imaging arm's X-ray source and/or X-ray detector. The light source can be positioned to direct its light to a field of view that is located between the X-ray source and the X-ray detector. The light can comprise a light source, a switching mechanism, and wiring to connect the light source and switching mechanism to a power source, such as a battery. The light can be electrically connected into the imaging arm or can be part of an accessory that can be retrofitted to the imaging arm.

### BRIEF DESCRIPTION OF THE DRAWINGS

The following description can be better understood in light of the Figures, in which:

FIG. 1 shows a side schematic view of some embodiments of a C-arm comprising some embodiments of an X-ray imaging arm light that is disposed near an X-ray detector;

FIG. 2 shows a side perspective view of some embodiments of the X-ray imaging arm light;

FIG. 3 shows a side schematic view of a C-arm comprising some embodiments of the X-ray imaging arm light that is disposed near an X-ray source;

FIG. 4 shows a side schematic view of a C-arm comprising some embodiments of the X-ray imaging arm light that is disposed at both the X-ray detector and the X-ray source;

FIG. 5 shows a face plan view of some embodiments of the X-ray imaging arm light comprising a structural member;

FIG. 6 shows a side perspective view of some embodiments in which the field of view light comprises a square shaped structural member;

FIG. 7 shows a front perspective view of some embodiments of the X-ray imaging arm light comprising a clamping mechanism and a laser aimer; and

FIG. 8 shows a back perspective view of some embodiments of the X-ray imaging arm light comprising the clamping mechanism and the laser aimer.

The Figures illustrate specific aspects of the described X-ray imaging arm lights and methods for making and using such lights. Together with the following description, the Figures demonstrate and explain the principles of the structures, methods, and principles described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices.

### DETAILED DESCRIPTION

The following description supplies specific details in order to provide a thorough understanding. Nevertheless, the skilled artisan will understand that the described X-ray imaging arm lights and associated methods of making and using the lights can be implemented and used without employing these specific details. Indeed, the lights and associated methods can be placed into practice by modifying the described devices and methods and can be used in conjunction with any other apparatus and techniques conventionally used in the industry. For example, while the description below focuses on methods for making and using the X-ray imaging arm lights with a C-arm positioning device, the described field of view lights can be used with any suitable X-ray device, including a mini C-arm, Compact style C-arm, and/or a standard-sized C-arm device.

The present application describes an X-ray imaging arm light. In some embodiments, this light illuminates a field of view that is located between an X-ray source and an X-ray detector on an X-ray imaging arm (such as a C-arm or an O-arm) and so can be referred to as a field of view light. The field of view light can reduce shadows that appear as the imaging arm is repositioned and blocks ambient light, diminish shadows that appear as an operator of the imaging arm (or another person) blocks ambient light, and help eliminate poor lighting conditions. The field of view light can make it easier for the operator of the imaging arm to see the object being X-rayed, can improve the X-ray process, and can improve the quality of the X-ray images taken with the imaging arm.

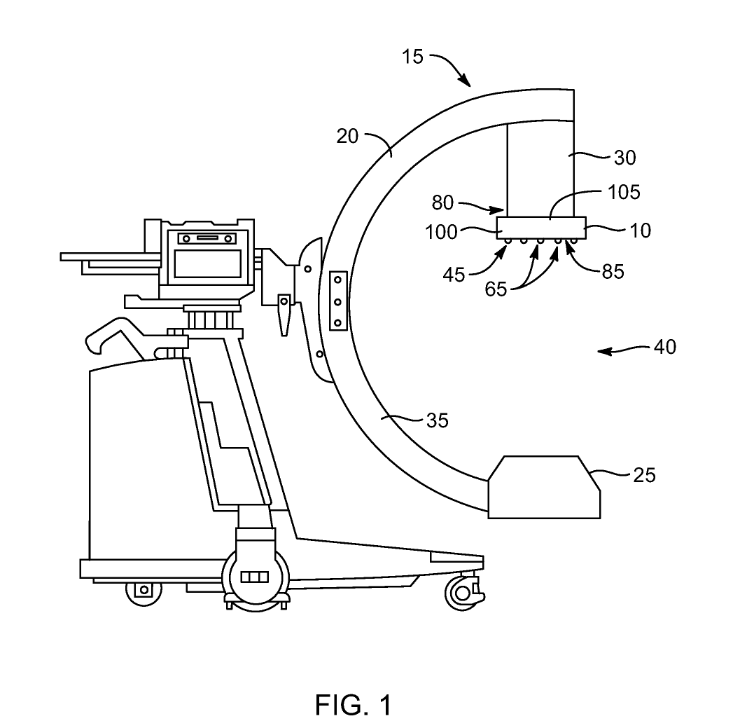

The X-ray imaging arm light can be used with any suitable X-ray imaging arm that can be used to take X-ray images of an object to be analyzed (i.e., a portion of a patient's body). For example, the imaging arm can comprise a mini C-arm, a standard C-arm, a compact style C-arm, and/or an O-arm. By way of illustration, FIG. 1 shows some embodiments in which X-ray imaging arm field of view light 10 is used with an X-ray imaging arm 15 that comprises a standard C-arm 20.

While the imaging arm 15 can comprise any suitable component for X-ray operation, FIG. 1 shows some embodiments in which the imaging arm 15 comprises an X-ray source 25 and an X-ray detector 30 that are connected together by an elongated structural member 35. FIG. 1 shows that the elongated structural member 35 holds the X-ray source 25 and X-ray detector 30 in relation to each other so that the source and detector face each other and so that they are spaced apart to define a gap 40 that is large enough to allow a portion of a patient's body (e.g., a limb, an extremity, etc.) to be inserted into the path of the X-ray beam (not shown) for X-ray imaging.

The imaging arm 15 can comprise any suitable X-ray source 25 and X-ray detector 30 that can be respectively disposed at nearly opposite positions on the elongated structural member 35 so as to face each other and allow the imaging arm to take X-ray images. In this regard, the X-ray source can comprise a standard X-ray source, a fixed X-ray source, a rotating anode X-ray source, and/or a fluoroscopic X-ray source. Moreover, the X-ray detector can comprise any suitable known or novel X-ray detector, such as an image intensifier, a fixed X-ray detector, and/or a digital flat panel detector.

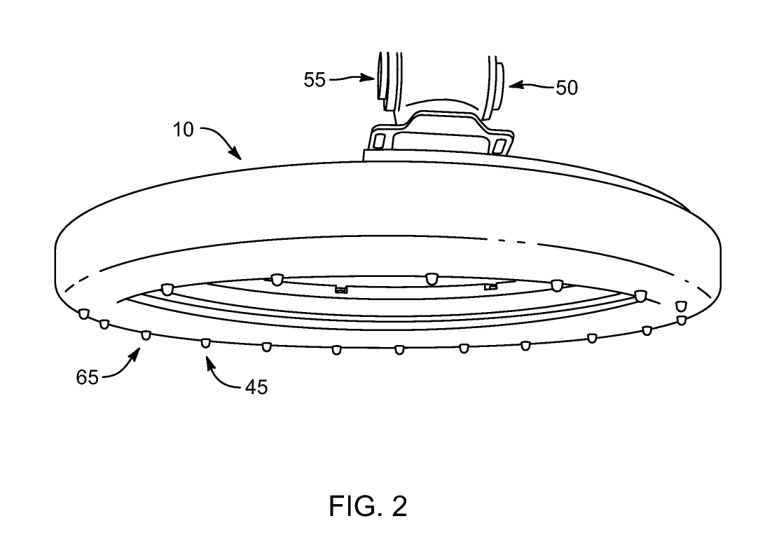

The X-ray imaging arm field of view light 10 can comprise any suitable component that allows the light to illuminate a field of view disposed between the X-ray source 25 and the X-ray detector 30. FIG. 2 shows some embodiments in which the field of view light 10 comprises one or more light sources 45, switching mechanisms 50, power sources 55, and electrical connectors 60 (not shown).

The light source 45 can comprise any suitable light generating device by which electrical energy can be converted into optical energy in order to light the field of view disposed between the X-ray source 25 and the X-ray detector 30. For instance, the light source can comprise an incandescent light bulb, a light emitting diode (“LED”), a non-laser light source, a halogen light, a compact fluorescent bulb, a Cold Cathode Fluorescent lamp (CCFL) assembly, and/or combinations thereof. Indeed, FIG. 2 shows some embodiments in which the light source 45 comprises multiple LEDs 65.

The light source 45 can comprise virtually any suitable number of light generating devices (e.g., LEDs 65). For instance, the light source can comprise 1, 2, 3, 4, 5, 6, 7, 8, 9, 10, or even more light generating devices. By way of illustration, FIG. 2 shows some embodiments in which the field of view light 10 comprises multiple LEDs 65.

The light generating devices (e.g., LEDs 65) of the light source 45 can be configured to direct their light from a perimeter of the X-ray source 25 and/or the X-ray detector 30 to a field of view in the gap 40 between these two components. The light generating devices may produce any suitable color temperature, including white light, yellow light, red light, etc. In some embodiments, the light generating devices emit a substantially white light. In other embodiments, the light generating devices can produce a single color or multiple colors. For example, the light source can comprise a combination of different color light generating devices. One or more of the light generating devices can be compound light generating devices (e.g., a bi-color or tri-color light generating device).

The switching mechanism 50 can comprise any component capable of being used to selectively increase (e.g., turn on) and decrease (e.g., turn off) the power to the light source 45. Some examples of suitable switching mechanisms include a push-button switches, toggle switches, knife switches, slide switches, tactile-membrane switches, single-pull single-throw switches, twist switches, foot switches, a dimmer switch, and/or combinations thereof. FIG. 2 shows some embodiments in which the switching mechanism 50 comprises a push-button switch 70.

The switching mechanism 50 can comprise any number of switches that allow the light source to be turned on or off. By way of example, the switching mechanism may comprise one switch for a first set of one or more light generating devices, and a second switch for a second set of one or more light generating devices. Accordingly, the switching mechanism can allow its operator to turn on, turn off, dim, brighten, or otherwise control any suitable number of light generating devices in the light source 45.

The switching mechanism 50 can also be disposed in any suitable location that allows the operator to access it. By way of example, the switching mechanism can be disposed on or adjacent to the field of view light 10, the imaging arm (e.g., along with or separate from the imaging arm controls), a foot switch, a remote control, and/or on an imaging arm support structure (e.g., a mobile cart) (not shown).

The power source 55 can comprise any power source capable of providing sufficient energy to allow the light source 45 to emit a desired amount of light. Some examples of suitable power sources include any suitable form of battery, direct connection to the X-ray device power supplies, uninterruptable power supply, electrical outlets, and/or combinations thereof. In some embodiments where the field of view light 10 is retrofitted to the imaging arm 15 (as described below), FIG. 2 shows that the power source 55 can comprise a battery pack 75 containing one or more batteries (not shown). In other embodiments, however, the field of view light can be electrically connected to the imaging arm. In these other embodiments, the field of view light can receive its power from the power source that operates the imaging arm (e.g., an outlet connected to a local power grid, an internal battery supply, etc.) or any other power source.

The electrical connectors 60 can comprise any type of electrical connectors that electrically connect the light source 45 and/or switching mechanism 50 to the power source 55. Some examples of suitable electrical connectors comprise one or more wires, light sockets/connectors, electrical circuits, MOLEX type connectors, ribbon cables, traces on a printed circuit board, and/or combinations thereof. The electrical connector can connect the light source to the power source in any suitable manner, including by connecting the various light generating devices in series and/or in parallel.

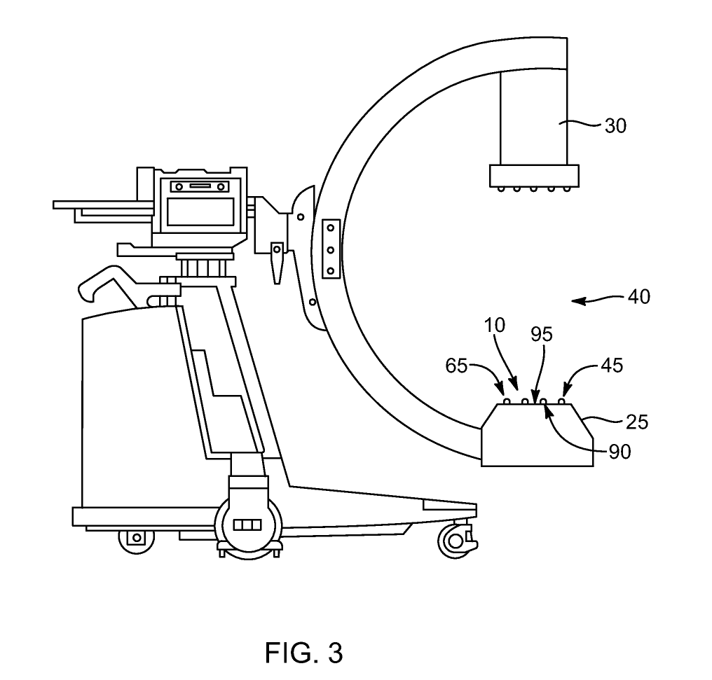

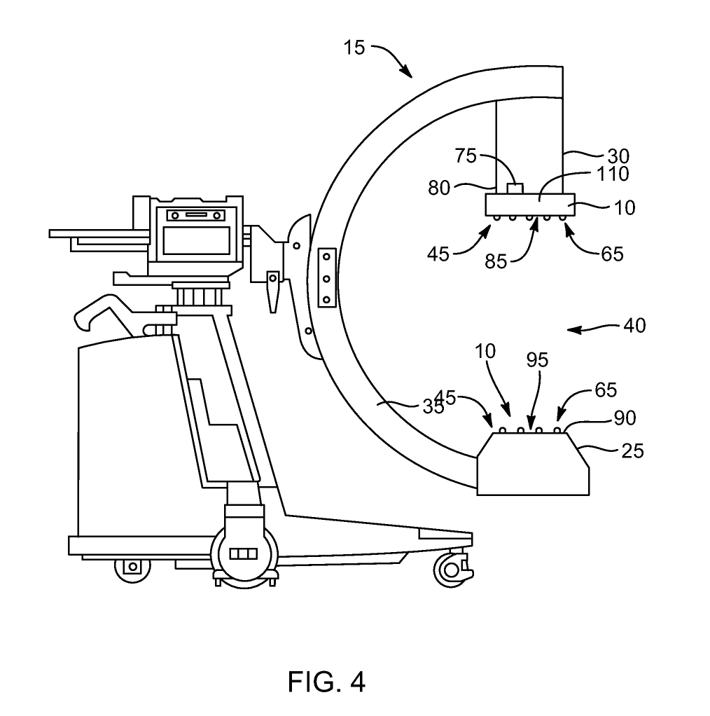

The light source 45 of the field of view light 10 can be disposed in any position that allows it to illuminate a field of view in the gap 40 between the X-ray source 25 and the X-ray detector 30. In some embodiments, the light source can be disposed at or adjacent (e.g., near, around at least a portion of a perimeter of, distal to, or proximal to) the X-ray source, and/or the X-ray detector. FIG. 1 shows some embodiments in which the light source 45 (e.g., LEDs 65) can be disposed about a perimeter 80 of a face 85 of the X-ray detector 30. In such embodiments, the light source may be able to illuminate the field of view in the gap without shining in the operator's eyes. FIG. 3 shows some embodiments in which the light source 45 (e.g., LEDs 65) can be disposed about a perimeter 90 of a face 95 of the X-ray source 25. FIG. 4 shows some embodiments in which the light source 45 (e.g., LEDs 65) can be disposed about a perimeter (e.g., perimeters 80 and 90) of a face (e.g., faces 85 and 95) of both the X-ray source 25 and the X-ray detector 30. In some configurations, the light source could be placed proximate or even on the X-ray source, especially if the light source were used with a mini C-arm.

The field of view light 10 can be connected to the imaging arm 15 in any suitable manner that allows the light to illuminate a field of view in the gap 40 between the X-ray source 25 and the X-ray detector 30. For example, the field of view light can be electrically connected to the imaging arm, permanently fastened to the imaging arm, and/or removably fastened to the imaging arm. The field of view 10 light can be connected to the imaging arm 15 in any configuration that allows the light to function as explained herein. In one example, FIG. 4 shows that the light source 45 (e.g., LEDs 65) is integrally attached to or embedded in the imaging arm 15 (e.g., the X-ray source 25). While FIG. 4 shows that the light source 45 (e.g., LEDs 65) can extend past a face (e.g., face 95) on the imaging arm 15, in other embodiments the light source can be flush with and/or below such a face.

The field of view light 10 can be permanently or removably attached to the imaging arm as a retrofitted (or add-on) component. By way of illustration, FIG. 1 shows some embodiments in which the field of view light 10 comprises the light source 45, which, in turn, is attached to a structural member 100 that can be connected (i.e., being retrofitted) to the imaging arm 15. Some examples of suitable structural members include a protective bumper, a ring, an elongated support, threaded holes (screws), a clamp, and/or any other member (or combination of members) that is capable of disposing the light source at a perimeter of a face (e.g., face 85 and/or face 95) of the X-ray source 25 and/or X-ray detector 30. FIG. 1 shows some embodiments where the structural member 100 comprises a protective image arm bumper 105.

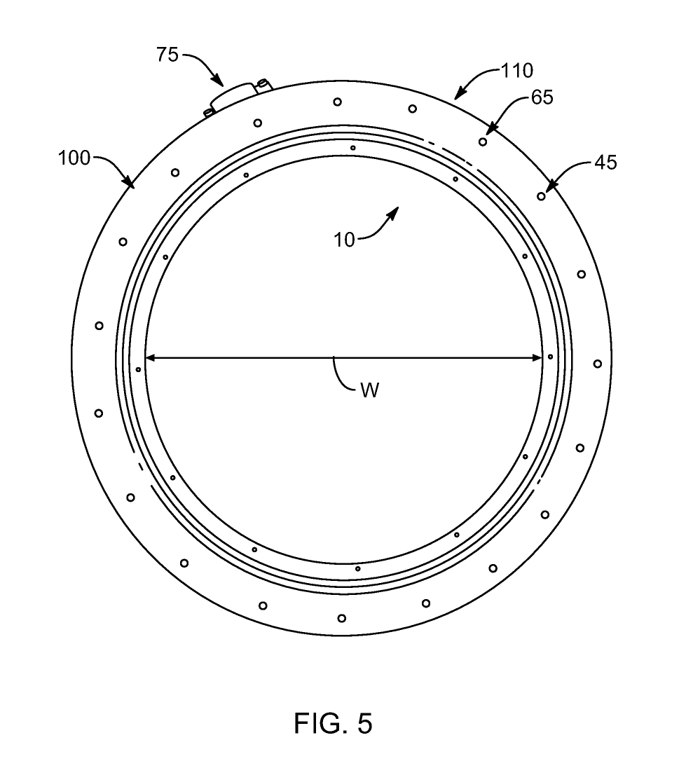

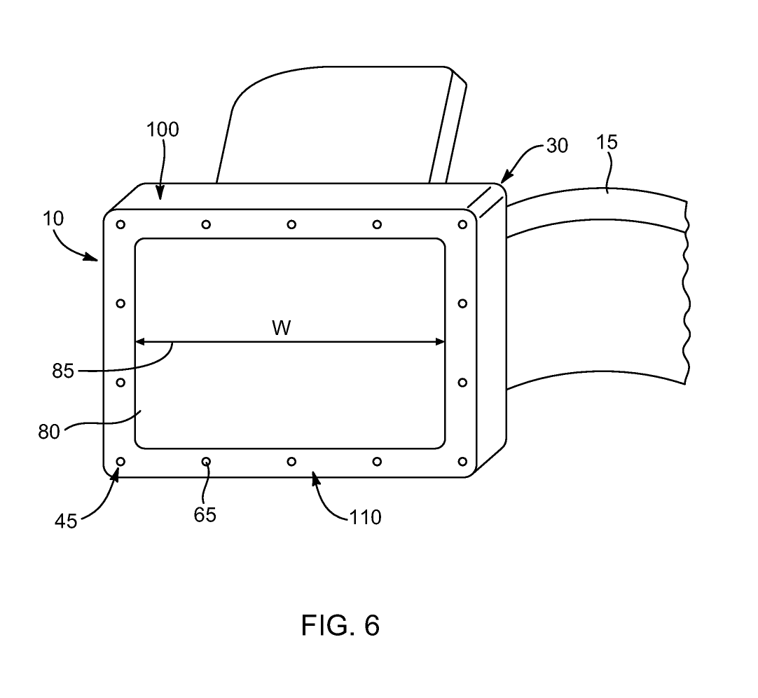

The structural member 100 can have any configuration that supports the light 10 and allows it to dispose the light source 45 at a perimeter of the X-ray source 25 and/or X-ray detector 30. In some embodiments, FIG. 5 shows that the structural member 100 can have a substantially circular appearance (from its face view). In other embodiments, the structural member can have any other suitable shape, including a square, rectangular, straight, ellipsoidal, polygonal, irregular, and/or any other shape. FIG. 6 shown even other embodiments in which the structural member 100 has a substantially square appearance (e.g., when it is disposed around a digital flat panel detector 99).

In another configuration, the structural member can comprise an imaging arm bumper, Some examples of imaging arm bumpers include an L-shaped cushioned bumper, a fluid filled bladder made from silicon rubber, a bumper formed from a viscoelastic material (e.g., a foam), a bumper comprising a fluid (e.g., air, water, etc.) and having a fluid tight outer membrane, a bumper comprising a pressure sensing device and switch to cut power to an imaging arm positioning device (not shown), or combinations thereof. FIG. 5 shows some embodiments in which the light source 45 (e.g., LEDs 65) can be disposed in an air filled bumper 110 having the power source (e.g., battery pack 75) attached thereto.

The structural member can have any inner diameter or width W that allows it to dispose the light source 45 at a perimeter of the X-ray source 25 and/or X-ray detector 30 for the X-ray equipment it is used with. In some embodiments, the structural member can have an inner diameter that is selected from a width between about 2 and about 20 inches, between 4 and about 15, between about 8 and about 13, and between about 9 and about 12 inches. In other embodiments, the structural member has an inner diameter that is about 9 inches±0.5 inches. In still other embodiments, however, the structural member has an inner diameter that is about 12 inches±0.5 inches. Accordingly, the field of view light can be retrofitted to many existing C-arms that are currently in use (i.e., C-arms having an image intensifier with an outer diameter at its face 85 that is about 9 or about 12 inches).

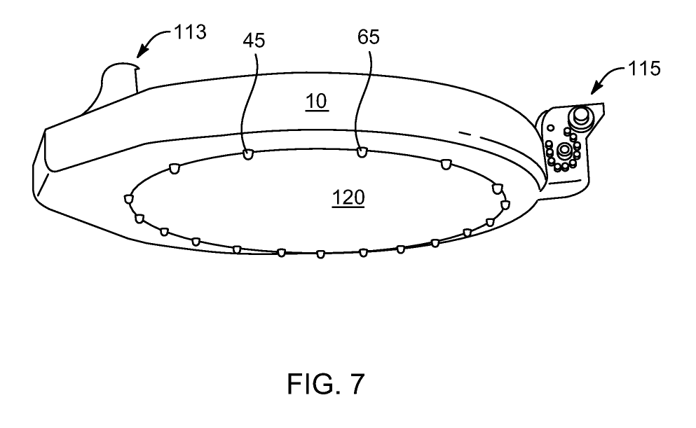

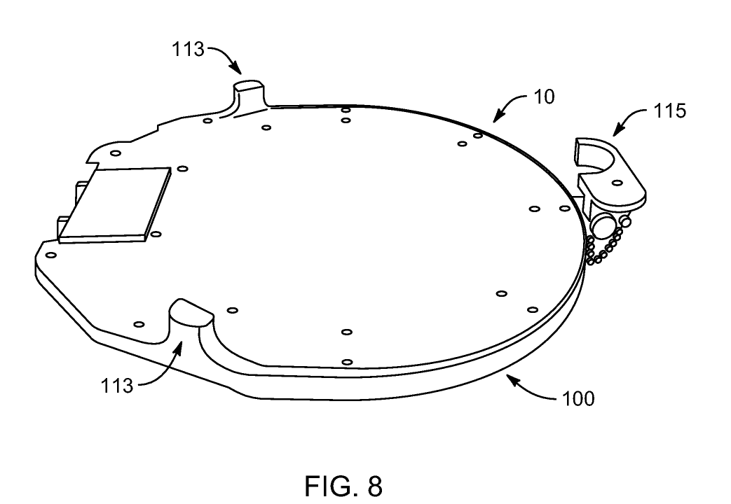

The structural member 100 can be temporarily or permanently connected to the imaging arm in any suitable manner. In some embodiments, the structural member can be connected to the imaging arm through the use of one or more adhesives, latches, straps, clamps, catches, cam lever with securing locking pin, frictional fittings, mechanical connectors, or combinations thereof. FIGS. 7 and 8 show some embodiments in which the structural member 100 comprises a plurality of catches 113 and a cam lever 115 that is configured to connect the structural member to the imaging arm (e.g., adjacent to the X-ray source 25 and/or the X-ray detector 35).

The X-ray imaging arm field of view light 10 can be combined with other components used in x-ray equipment. In some embodiments, the field of view light can be combined with a conventional laser aimer. In this manner, both the laser aimer and the field of view light can be available in a single imaging arm 15 or structural member 100. FIGS. 7 and 8 shows some embodiments in which the field of view light 10 comprises the structural member 100 having the light source 45 (e.g., LEDs 65) and a laser aimer 120, which can help the operator determine the proper position (with respect to the X-ray source 25 and the X-ray detector 30) of the object (e.g., body part) that is being imaged.

The X-ray imaging arm field view light 10 can be made in any manner that forms the structures described herein. By way of example, the light can be formed through a process involving molding, extruding, injection, casting, cutting, stamping, bending, drilling, bonding, welding, soldering, electrically connecting, mechanically connecting, and/or any other suitable process.

The described X-ray imaging arm field view light 10 can also be used in any suitable manner. By way of example, an operator can turn the light source 45 on to properly visualize the object that is being imaged and then turn off the light source after the object has been imaged. In another example, if a surgeon is operating in conjunction with an X-ray system where the C-arm is located over the patient while operating, the light can aid the surgeon's actions by providing additional light to the workspace (i.e., more light in the body cavity or, in the case of non-invasive procedures, on the surface of the body).

The X-ray imaging arm field view light 10 has several features. First, the light may be able to greatly illuminate the object being X-rayed in order to improve the speed, accuracy, and quality of the X-ray process. Second, because the field of view light 10 can be attached to a structural member 100, the light can be retroactively fitted or added on to existing imaging arms that do not previously contain them. Third, because the field of view light 10 can be used with a laser aimer, the light can be retrofit to an imaging arm to give the arm better viewing and aiming capabilities.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. An X-ray imaging light accessory, comprising:
a structural member configured to substantially fit at a perimeter of an X-ray source or an X-ray detector; and
a light source disposed on the structural member so that the light source directs light to a field of view between the X-ray source and the X-ray detector when the structural member is attached at the perimeter of the X-ray source or the X-ray detector, wherein the light source is coupled to a power source.

2. The accessory of claim 1, further comprising an attachment mechanism to attach the structural member near the perimeter of the X-ray source or the X-ray detector.

3. The accessory of claim 1, wherein the structural member removably attaches near the perimeter of the X-ray source.

4. The accessory of claim 1, wherein the structural member removably attaches near the perimeter of the X-ray detector.

5. The accessory of claim 1, wherein the light source comprises a plurality of LEDs.

6. The accessory of claim 1, wherein the power source comprises a battery.

7. The accessory of claim 1, wherein the power source comprises a power source of the X-ray imaging system.

8. The accessory of claim 1, wherein the light accessory further comprises a laser aimer.

9. The accessory of claim 1, wherein the structural member comprises a bumper.

10. The accessory of claim 1, wherein the X-ray detector comprises an image intensifier.

11. The accessory of claim 1, wherein the X-ray detector comprises a digital X-ray detector.

12. The accessory of claim 1, wherein the X-ray imaging system comprises a mobile X-ray fluoroscopic imaging system.

13. An X-ray imaging system, comprising:
an X-ray source and an X-ray detector disposed nearly opposite to each other; and
a light source disposed near a perimeter of the X-ray source or the X-ray detector, wherein the light source is positioned to direct light to a field of view on an object being imaged by the X-ray imaging system.

14. The imaging system of claim 13, wherein the light source is attached to a structural member that is retrofitted to the imaging system.

15. The imaging system of claim 13, wherein the light source is electrically coupled to the imaging system.

16. The imaging system of claim 14, wherein the structural member further comprises a laser aimer.

17. The imaging system of claim 13, wherein the light source removably attaches near the perimeter of the X-ray source.

18. The imaging system of claim 13, wherein the light source is embedded adjacent to the perimeter of the X-ray source.

19. The imaging system of claim 13, wherein the light source removably attaches near the perimeter of the X-ray detector.

20. The imaging system of claim 13, wherein the light source is embedded near the perimeter of the X-ray detector.

21. The imaging system of claim 13, wherein the light source comprises a plurality of LEDs.

22. An X-ray imaging system, comprising:
an X-ray imaging arm comprising an X-ray source and an X-ray detector disposed at nearly opposing locations of the imaging arm; and
a light source comprising multiple lights that are embedded near a perimeter of the X-ray source or the X-ray detector, wherein the light source is positioned to direct light to a field of view between the X-ray source and the X-ray detector, and wherein the light source is electrically coupled to the imaging arm.

23. The imaging system of claim 22, wherein the light source is disposed near the perimeter of both the X-ray source and the X-ray detector.

24. The imaging system of claim 22, wherein the light source further comprises a dimming mechanism.

25. The imaging system of claim 22, wherein the light source is disposed near the perimeter of the X-ray detector.
